(19) United States
(12) Patent Application Publication
(10) Pub. No.: US 2025/0375155 A1
(43) Pub. Date: Dec. 11, 2025

(54) BIOLOGICAL INFORMATION PROCESSING DEVICE, BIOLOGICAL INFORMATION PROCESSING METHOD, AND NON-TRANSITORY STORAGE MEDIUM STORING PROGRAM THEREOF

(71) Applicant: FUKUDA DENSHI CO., LTD., Tokyo (JP)

(72) Inventors: Mai KOBAYASHI, Tokyo (JP); Ryotaro HASHIMOTO, Tokyo (JP); Tatsuya YONEYAMA, Tokyo (JP)

(73) Assignee: FUKUDA DENSHI CO., LTD., Tokyo (JP)

(21) Appl. No.: 19/230,123

(22) Filed: Jun. 6, 2025

(30) Foreign Application Priority Data
Jun. 7, 2024 (JP) 2024-093191

(51) Int. Cl. A61B 5/00; A61B 5/0205; A61B 5/024; A61B 5/08; A61B 5/145

(52) U.S. Cl. CPC A61B 5/4818; A61B 5/0205; A61B 5/02405; A61B 5/7203; A61B 5/7278; A61B 5/7282; A61B 5/0816; A61B 5/14542

(57) ABSTRACT

A biological information processing device detects a hypopnea or apnea interval in a period of measurement of a pulse wave during sleep. Also, the biological information processing device determines presence or absence of a respiration effort on the basis of the detected pulse wave in the hypopnea or apnea interval. The biological information processing device identifies a type of respiration disorder for the detected hypopnea or apnea interval on the basis of the presence or absence of the respiration effort.

### CROSS REFERENCE TO PRIOR APPLICATION

This application claims priority to Japanese patent application no. 2024-093191 (filed on Jun. 7, 2024), which is hereby incorporated by reference in its entirety.

### BACKGROUND

The present disclosure relates to a biological information processing device, a biological information processing method, and a program, and particularly relates to a technology of evaluating sleep of a subject.

Among sleep disordered breathing (SDB), a disease satisfying a criterion such as one that the number of times of apnea or hypopnea is five per hour or more is referred to as sleep apnea syndrome (SAS). SAS is roughly classified into obstructive sleep apnea (OSA) and central sleep apnea (CSA). In addition, a mixture of OSA and CSA is referred to as mixed sleep apnea (MSA).

OSA and CSA can be distinguished from each other by the presence or absence of a respiration effort (movement of a thorax and an abdominal wall) at a time of apnea or hypopnea. OSA is a state in which a respiration effort is observed, but a normal respiration is hindered by the obstruction of an upper airway. In contrast, CSA is caused by a respiratory center and is therefore a state in which a respiration effort itself is not observed and a normal respiration is hindered.

OSA and CSA have different causes, and thus different treatment methods are used therefor. It is therefore desired to distinguish between OSA and CSA for the diagnosis of SAS. The diagnosis of SAS necessitates an overnight polysomnography (PSG) test. However, the overnight polysomnography test usually necessitates lodging at a hospital and necessitates wearing a large number of sensors. Hence, a relatively heavy burden is imposed on the subject. A simpler screening test (referred to also as a simple SAS test, a simple PSG test, or the like) than the PSG test is therefore often performed. Japanese Patent No. 5969283 discloses a portable sleep evaluating device for the screening test.

### SUMMARY

In the past, both the PSG test and the simple PSG test have necessitated the use of a belt-shaped respiratory movement sensor that is attached to at least one of a thoracic region and an abdominal region to distinguish between OSA and CSA. The respiratory movement sensor detects movement of the thorax or the abdominal wall on the basis of a change in inductance, a change in voltage generated by a piezoelectric film, or the like as a belt extends and contracts.

However, because the respiratory movement sensor is in the shape of a belt, the wearing position of the respiratory movement sensor tends to be moved upward or downward due to body movement during sleep. It is therefore not easy to detect respiratory movement stably during sleep. This is especially true in a case where separate respiratory movement sensors are attached to both the abdominal region and the thoracic region. Particularly in a case of a simple PSG test in which a person not engaged in medical profession such as the subject or a family member thereof needs to wear the respiratory movement sensor, the length adjustment and the wearing position of the sensor tend to be inappropriate, so that this problem becomes greater.

The present disclosure has been made in view of the problems of such a related-art technology. As a part of modes of the present disclosure, the present disclosure provides a biological information processing device, a biological information processing method, and a program that can identify the type of respiration disorder in sleep apnea or hypopnea without the use of respiratory movement sensors that are worn on the abdominal region and the thoracic region.

In one mode of the present disclosure, the present disclosure provides a biological information processing device including an obtainer configured to obtain a pulse wave during sleep, a detector configured to detect a hypopnea or apnea interval in a period of measurement of the pulse wave, a determiner configured to determine presence or absence of a respiration effort on the basis of the pulse wave in the hypopnea or apnea interval, and an identifier configured to identify a type of respiration disorder for the hypopnea or apnea interval on the basis of the presence or absence of the respiration effort.

With such a configuration, according to the present disclosure, it is possible to provide a biological information processing device, a biological information processing method, and a program that can identify the type of respiration disorder in sleep apnea or hypopnea without the use of respiratory movement sensors that are worn on the abdominal region and the thoracic region.

### BRIEF DESCRIPTION OF THE DRAWINGS

FIG. 1 is a diagram illustrating an example of a functional configuration of a simple PSG test device as an example of a biological information processing device according to an embodiment;

FIG. 2 is a flowchart of assistance in explaining an example of operation of the simple PSG test device according to the embodiment;

FIG. 3 is a flowchart of assistance in explaining an example of operation of the simple PSG test device according to the embodiment;

FIGS. 4A-4C are diagrams of assistance in explaining a specific example of operation of the simple PSG test device according to the embodiment;

FIG. 5 is a flowchart of assistance in explaining an example of operation of the simple PSG test device according to the embodiment; and

FIG. 6 is a diagram of assistance in explaining a specific example of operation of the simple PSG test device according to the embodiment.

### DETAILED DESCRIPTION OF THE PREFERRED EMBODIMENT

The present disclosure will hereinafter be described in detail on the basis of an illustrative embodiment thereof with reference to the accompanying drawings. It is to be noted that the following embodiment does not limit the disclosure according to claims. In addition, while a plurality of features are described in the embodiment, not all of the plurality of features are necessarily essential to the disclosure, and the plurality of features may be optionally combined with each other. Further, in the accompanying drawings, identical or similar configurations are identified by the same reference signs, and repeated description thereof will be omitted.

Incidentally, in the following, a description will be made of a mode in which the present disclosure is carried out in a simple PSG test device as an example of a biological information processing device. However, functions of measuring and recording biological information are not essential in the present disclosure, and can be performed in optional electronic equipment including one or more arithmetic circuits or processors. Such electronic equipment includes computer equipment (a personal computer, a tablet computer, a media player, a smart phone, a smart watch, and the like). These are illustrative, and the present disclosure can also be carried out in other electronic equipment. That is, computer equipment separate from the PSG test device which computer equipment analyzes data recorded by the PSG test device is also included in the biological information processing device according to the present disclosure.

The present disclosure will hereinafter be described in detail on the basis of an illustrative embodiment thereof with reference to the drawings.

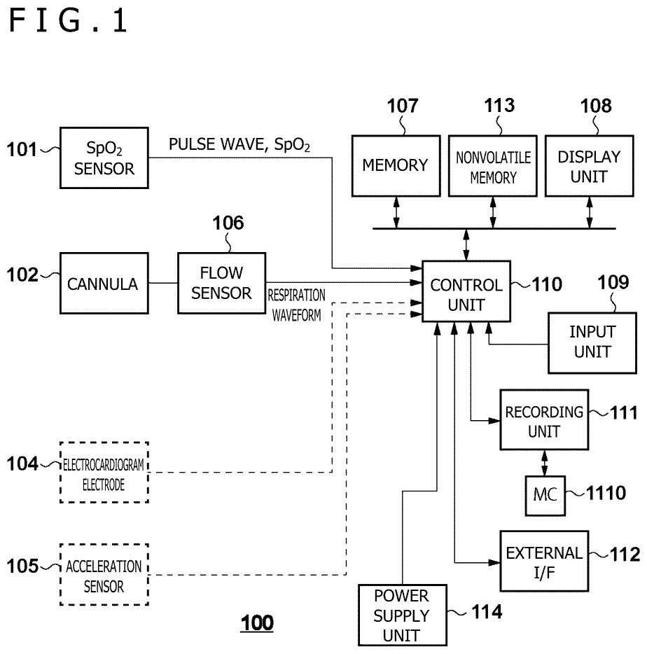

FIG. 1 is a block diagram illustrating an example of a functional configuration of a simple PSG test device 100 as an example of the biological information processing device according to the embodiment of the present disclosure. The biological information in the present specification refers to information indicating the state or activity of a living body. The biological information includes information directly detected as an electric signal as in an electrocardiogram or an electromyogram and information obtained by converting, by a sensor or the like, the state or activity of the living body such as arterial oxygen saturation (SpO₂), body temperature, respiration, a pulse wave, a snore, or body movement into data or a signal that can be handled by the device.

### (Configuration)

In the simple PSG test device 100 according to the present embodiment, an SpO₂ sensor 101 is a pulse oximeter that measures the SpO₂. The SpO₂ sensor 101 measures SpO₂ from one set of photoelectric pulse waves detected by using red light and infrared light. The SpO₂ sensor 101 therefore functions also as a pulse wave sensor. The SpO₂ sensor 101 continuously outputs a measured pulse wave and SpO₂. As a matter of course, the present disclosure is not limited to including a pulse oximeter that can detect the pulse wave and SpO₂ as in the present embodiment. For example, the present disclosure may include equipment capable of detecting a pulse wave and equipment capable of detecting SpO₂ independently of each other, and may include a smart watch or the like which is provided with an SpO₂ sensor or the like rather than a pulse oximeter.

A cannula 102 has an opening in the vicinity of a nostril of a subject, and is connected to a main unit of the simple PSG test device 100 via a flow sensor 106. The flow sensor 106 outputs an electric signal (respiration waveform) corresponding to an air flow within a tube connected to the cannula 102. Incidentally, the respiration waveform to be detected may be an oral respiration waveform. In addition, any publicly known sensor such as a pressure sensor or a temperature sensor can be used for the flow sensor 106.

Electrocardiogram electrodes 104 are, for example, electrodes for detecting a unipolar or bipolar two-channel electrocardiogram signal. An acceleration sensor 105 is a sensor for determining the body position (for example, a right side decubitus position, a left side decubitus position, a supine position, a prone position, or an upright position) of the subject and detecting body movement thereof. Incidentally, the electrocardiogram electrodes 104 and the acceleration sensor 105 are not essential, and are indicated by broken lines in FIG. 1. Incidentally, in a case where the acceleration sensor 105 is used, it is possible to determine from a measurement result of the acceleration sensor 105 whether the subject is asleep or awake. Therefore, a reduction in a load on analysis processing and an improvement in accuracy can be achieved by, for example, excluding, from a target of the analysis processing, data during wakefulness among pieces of data regarding the pulse wave or the like recorded during the test.

A memory 107 is a memory used for work by a control unit 110. The control unit 110 loads a program into the memory 107, and executes the program. The control unit 110 also uses the memory 107, for example, as a buffer for temporarily storing the data regarding the biological information measured or read out from a memory card 1110, or uses the memory 107 as a video memory of a display unit 108.

A nonvolatile memory 113 stores the program executed by the control unit 110, graphical user interface (GUI) data of a menu screen and the like, set values of the simple PSG test device 100, and the like. The nonvolatile memory 113 is electrically rewritable.

The display unit 108 is a liquid crystal display (LCD), for example. The display unit 108 displays operation conditions of the simple PSG test device 100, the measured biological information, user information, a GUI such as the menu screen, and the like.

The control unit 110 is one or more programmable processors (hereinafter a CPU(s)), for example. The control unit 110 controls the operation of various parts of the simple PSG test device 100 and implements various functions of the simple PSG test device 100 including operations to be described later, by loading the program stored in the nonvolatile memory 113 into the memory 107 and executing the program.

An input unit 109 is a general term of a plurality of input devices which are provided to the simple PSG test device 100 and which are used by a user (a medical worker and the subject) to input various kinds of instructions and settings. The input unit 109 can include not only input devices necessitating a physical operation, such as a switch, a button, a dial, and a touch panel, but also input devices not necessitating a physical operation, such as input equipment configured to recognize an instruction by voice recognition. An instruction input through the input unit 109 is detected by the control unit 110, and the control unit 110 performs an operation according to the instruction.

Incidentally, an audio outputter such as a speaker may be provided and configured to notify the user of an operation state of the device, the occurrence of an error, an operation procedure, and the like by sound or the like.

A recording unit 111 records the measured biological information data in the memory card (MC) 1110 as an example of a recording destination and reads the biological information data recorded in the memory card 1110 according to control of the control unit 110. The biological information data may be recorded on another removable recording medium or a nonremovable recording medium.

An external I/F 112 is an interface for wire or wireless communication with an external device such as a personal computer. There is no particular limitation on the kind of connectable external equipment and a protocol of communication with the external equipment. The external equipment may be equipment on a network accessible through the external I/F 112 (for example, a cloud server) or the like.

A power supply unit 114 includes a primary battery or a secondary battery, for example. The power supply unit 114 supplies power to various parts including the control unit 110. The control unit 110 can measure the power supply voltage (battery voltage) of the power supply unit 114. The power supply unit 114 may use a commercial power supply through an AC adapter or the like.

### (Recording Operation)

A description will next be made of an operation of recording biological information data in the simple PSG test device according to the present embodiment. Incidentally, an overall operation at a time of recording is intended to be described here. Thus, suppose that at least the SpO₂ sensor 101 and the cannula 102 are worn on a predetermined body part of the subject and that a recording start condition is satisfied. Suppose that the electrocardiogram electrodes 104 and the acceleration sensor 105 are not connected.

When a recording start instruction is input from the input unit 109, the control unit 110 writes a recording start date and time in the memory 107, and starts an operation of measuring and recording biological information. The control unit 110 sequentially stores the pieces of data regarding a pulse wave and SpO₂ obtained from the SpO₂ sensor 101 in the memory 107.

In addition, the control unit 110 receives an electric signal (respiration waveform) output by the flow sensor 106 connected to the cannula 102, performs A/D conversion of the electric signal, and stores the electric signal as respiration waveform data in the memory 107. Incidentally, the control unit 110 may apply amplification processing and filter processing to the respiration waveform data, and thereby separate a component of trachea sound (snore) from the respiration waveform data.

The control unit 110 applies analysis processing to the biological information data (SpO₂, the pulse wave, and the respiration waveform) stored in the memory 107. In the analysis processing, the control unit 110 obtains detected hypopnea or apnea intervals, the duration and the number of detected hypopnea and/or apnea intervals, the presence or absence of a decrease in SpO₂ by predetermined percentage points (for example, 2 to 4 percentage points) or more, the duration and the number of decreases, a pulse rate (a maximum, a minimum, and an average), and the like. The control unit 110 also determines the type of respiration disorder (OSA, CSA, or MSA) for a detected hypopnea and/or apnea interval. Details of these pieces of processing will be described later.

The control unit 110 records the biological information data stored in the memory 107 as a data file for each predetermined unit time into the memory card 1110. In addition, the control unit 110 records the data of an analysis result into the memory card 1110.

The control unit 110 can also display an indication to the effect that recording is being performed, a measurement period (for how many hours the recording is to be performed), a planned recording end date and time, and the like on the display unit 108 during the recording. The control unit 110 may also display a state of attachment of a sensor or the like, a part of measured values, and the like during the recording.

The control unit 110 continues performing the measurement processing, the analysis processing, and the recording processing described above until determining that a recording ending condition is satisfied. The control unit 110 can determine that the recording ending condition is satisfied, for example, in a case where a recording time has reached a set automatic recording end time (for example, eight hours), in a case where input of a recording ending instruction through the input unit 109 is detected, or the like.

### (Sleep Evaluation Processing)

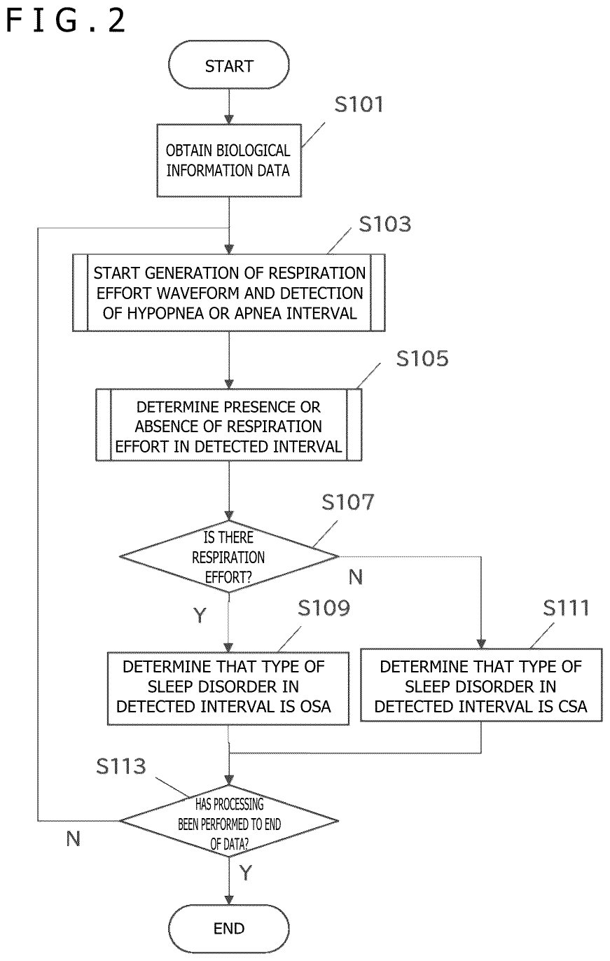

Next, sleep evaluation processing performed by the simple PSG test device (control unit 110) will be described with reference to a flowchart illustrated in FIG. 2. The sleep evaluation processing is processing of detecting a hypopnea or apnea interval and determining the type of respiration disorder. The control unit 110 can perform the sleep evaluation processing during the test (for example, as a part of the analysis processing in the recording operation described above). The control unit 110 can also perform the sleep evaluation processing after an end of the test (for example, on the biological information data recorded in the memory card 1110).

In S101, the control unit 110 obtains the biological information data regarding an evaluation target. The biological information data regarding the evaluation target may be biological information data temporarily stored in the memory 107 during the recording operation, or may be biological information data stored in the memory 107 after being read from the memory card 1110 or obtained from external equipment through the external I/F 112.

The control unit 110 obtains the pieces of data regarding SpO₂ and the pulse wave for a predetermined period of time as the biological information data, and further obtains the data regarding the respiration waveform as required.

In S103, the control unit 110 starts to apply hypopnea or apnea interval detection processing and respiration effort waveform generation processing to the obtained biological information data for the predetermined period of time. Details of the respiration effort waveform generation processing will be described later. Incidentally, in a case where already recorded biological information data is used and information regarding hypopnea intervals and apnea intervals is recorded, the hypopnea or apnea interval detection processing can be omitted.

The control unit 110 can detect, as a hypopnea or apnea interval, an interval during which an amplitude of the respiration waveform continues to be decreased by a predetermined threshold value or more with respect to an amplitude of a normal interval (interval not classified as a hypopnea and apnea interval). Specifically, the control unit 110 can detect an interval during which there is a decrease of 90% or more from the amplitude of the normal interval as an apnea interval, and detect an interval during which there is a decrease of 30% (or 50%) or more but less than 90% as a hypopnea interval. Whether to set a detection threshold value for the hypopnea interval at 30%, 50%, or another numerical value depends on a user setting, for example. The amplitude of the normal interval can be determined by a predetermined method such as an average value of the amplitude of the respiration waveform during the interval of a length that is a certain percentage or more of a measurement period.

In addition, in a case where the control unit 110 does not obtain the respiration waveform, the control unit 110 can detect, as a hypopnea and apnea interval, an interval during which SpO₂ continues to be decreased by a predetermined threshold value (for example, 3 to 4 percentage points) or more from the value of the normal interval. In this case, a time lag from the state of hypopnea or apnea to a decrease in the value of SpO₂ may be taken into consideration, and an interval dating back by a predetermined period of time from the interval during which the value of SpO₂ continues to be decreased may be detected as a hypopnea interval and an apnea interval. Here, in the case of using SpO₂, the hypopnea interval and the apnea interval are not distinguished from each other. However, the hypopnea interval and the apnea interval may be detected individually by using a threshold value for detecting hypopnea and a threshold value for detecting apnea. In the case of using the value of SpO₂ for the detection of the hypopnea interval and the apnea interval, the respiration waveform does not need to be obtained, and therefore an amount of data to be obtained can be reduced. Further, it suffices to attach only the pulse oximeter to the subject during the recording operation, and therefore there is an advantage of being able to reduce a burden on the subject as compared with a case of attaching the cannula.

Incidentally, it is to be noted that, because the unit of the measured value of SpO₂ is %, the threshold value (%) in the case of using SpO₂ is an absolute value. For example, when the SpO₂ value in the normal interval is 98% and the threshold value is 3%, an interval during which the SpO₂ value continues to be decreased to 95% or less is detected as a hypopnea and apnea interval. Incidentally, the above-described threshold value is an example, and another value may be used.

In S105, the control unit 110 determines the presence or absence of a respiration effort (movement of a thorax or an abdominal wall for taking air into lungs) of the subject on the basis of a respiration effort waveform for the hypopnea or apnea interval detected in S103. Details of the determination processing will be described later. The control unit 110 stores a result of the determination in the memory 107.

In S107, the control unit 110 determines whether or not it is determined in S105 that there is a respiration effort. The control unit 110 performs S109 when it is determined that there is a respiration effort. The control unit 110 performs S111 when it is not determined that there is a respiration effort.

In S109, the control unit 110 determines that the type of sleep disorder is OSA for the hypopnea or apnea interval detected in S103. The control unit 110 stores the determination result in the memory 107 in association with information regarding the hypopnea or apnea interval detected in S103. The control unit 110 thereafter performs S113.

In S111, the control unit 110 determines that the type of sleep disorder is CSA for the hypopnea or apnea interval detected in S103. The control unit 110 stores the determination result in the memory 107 in association with the information regarding the hypopnea or apnea interval detected in S103. The control unit 110 thereafter performs S113.

Incidentally, when there are an interval with a respiration effort and an interval without a respiration effort in one hypopnea or apnea interval, the control unit 110 can determine that the type of sleep disorder is MSA. When there are an interval with a respiration effort and an interval without a respiration effort in one hypopnea or apnea interval, the control unit 110 may determine that the type of sleep disorder is OSA or CSA according to a relation between the lengths of the interval with a respiration effort and the interval without a respiration effort, ratios of the interval with a respiration effort and the interval without a respiration effort to the one hypopnea or apnea interval, or the like.

The control unit 110 may determine that the type of respiration disorder is OSA, for example, when the interval with a respiration effort is longer than the interval without a respiration effort, when a ratio of the interval with a respiration effort to the hypopnea or apnea interval is equal to or higher than a first threshold value ratio, and when a ratio of the interval without a respiration effort to the hypopnea or apnea interval is lower than a second threshold value ratio. The first and second threshold values may, for example, be determined empirically or set by the user.

In addition, the control unit 110 may determine that the type of respiration disorder is CSA, for example, when the interval without a respiration effort is longer than the interval with a respiration effort, when the ratio of the interval without a respiration effort to the hypopnea or apnea interval is equal to or higher than a third threshold value ratio, and when the ratio of the interval with a respiration effort to the hypopnea or apnea interval is lower than a fourth threshold value ratio. The third and fourth threshold values may, for example, be determined empirically or set by the user.

In S113, the control unit 110 determines whether or not an end of the biological information data regarding the evaluation target has been reached (whether or not evaluation has been performed to the end of the data). The control unit 110 ends the sleep evaluation processing when determining that the end of the biological information data regarding the evaluation target has been reached. The control unit 110 repeats the application of the processing from S103 to data not yet evaluated when determining that the end of the biological information data regarding the evaluation target has not been reached.

The control unit 110 can perform other processing using the result of the sleep evaluation processing according to a purpose for which the sleep evaluation processing is performed. For example, in a case where the control unit 110 performs the sleep evaluation processing during the test, the control unit 110 can include the result of the sleep evaluation processing in the analysis result to be recorded in association with the measured biological information data. In addition, for example, in a case where the control unit 110 performs the sleep evaluation processing on the biological information data recorded in the memory card 1110 after the test (or recorded in advance by another equipment), the control unit 110 can generate a report in a predetermined format together with another evaluation result, and display the generated report on the display unit 108 or record the generated report in the memory card 1110.

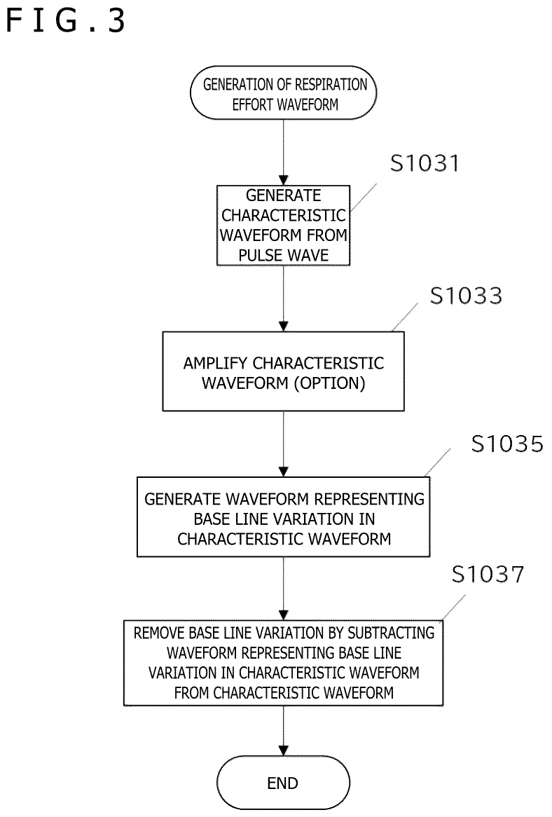

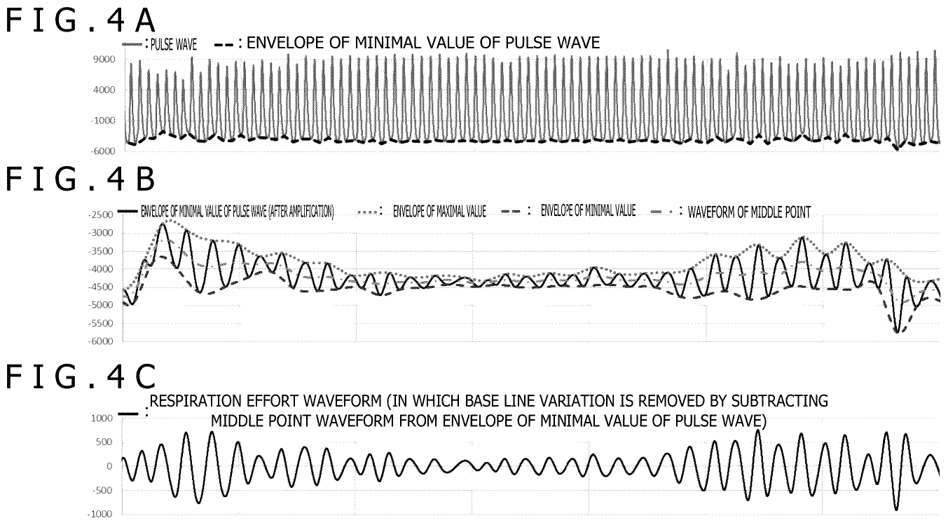

Next, referring to a flowchart illustrated in FIG. 3 and the diagrams illustrated in FIGS. 4A-4C, a description will be made of details of the respiration effort waveform generation processing started in S103.

In S1031, the control unit 110 generates a characteristic waveform from pulse wave data corresponding to a predetermined time. The characteristic waveform is information representing characteristics of the pulse wave. The characteristic waveform may be, for example, a waveform obtained on the basis of characteristic points of the pulse wave. The control unit 110 can generate, for example, any one of the following as the characteristic waveform.

(1) a waveform based on the characteristic points (for example, maximal values (top) or minimal values (bottom)) of an amplitude of the pulse wave (for example, a diagram obtained by connecting adjacent characteristic points or an envelope of the characteristic points)

(2) a waveform based on amplitude values (differences between maximal values and minimal values) of the pulse wave (for example, a diagram obtained by connecting amplitude values plotted in a coordinate system in which an amplitude value is indicated on an axis of ordinates and time is indicated on an axis of abscissas)

(3) a waveform based on time intervals of the pulse wave (for example, a diagram obtained by connecting time intervals plotted in a coordinate system in which the time intervals are indicated on an axis of ordinates and time is indicated on an axis of abscissas). A time interval of the pulse wave can be obtained as a time difference between characteristic points (maximal values, middle points, or minimal values of the amplitude) of the pulse wave at different pulses.

Here, suppose that, as illustrated in FIG. 4A, for example, the control unit 110 generates an envelope of minimal values (bottom) of the pulse wave as the characteristic waveform. The characteristic waveform based on the amplitude of the pulse wave may be a waveform obtained by any publicly known method from a point group plotted with an axis of abscissas indicating time and with an axis of ordinates indicating the amplitude (difference between the top and the bottom) after the amplitude of each pulse is sequentially obtained. The characteristic waveform based on time intervals of the pulse wave may be a waveform obtained by any publicly known method from a point group plotted with an axis of abscissas indicating time and with an axis of ordinates indicating the time intervals after a time interval between characteristic points of the same kind in the pulse wave of two different pulses (for example, pulses adjacent to each other) is sequentially obtained.

The control unit 110 samples the generated characteristic waveform at a predetermined frequency, and stores the sampled characteristic waveform as characteristic waveform data in the memory 107.

In S1033, the control unit 110 applies processing of amplifying an amplitude of the characteristic waveform to the characteristic waveform data stored in the memory 107. This amplification processing can be performed as required. The control unit 110 can determine an amplification factor such that, for example, a maximum amplitude of the characteristic waveform is a predetermined value. The control unit 110 stores the characteristic waveform data obtained by applying the amplification processing in the memory 107.

In S1035, the control unit 110 generates a waveform for removing base line variation in the characteristic waveform by using the characteristic waveform obtained by applying the amplification processing in S1033. Specifically, the control unit 110 can generate a waveform generated by using time series data regarding the characteristic points of the amplitude of the characteristic waveform (waveform obtained on the basis of the characteristic waveform) as the waveform for removing the base line variation in the characteristic waveform (that is, a waveform representing the base line variation in the characteristic waveform). The control unit 110 can generate, for example, any one of the following as the waveform representing the base line variation in the characteristic waveform. The control unit 110 stores the data of the generated waveform in the memory 107.

(1) a waveform based on maximal values of the characteristic waveform (for example, a diagram obtained by connecting maximal values adjacent to each other or an envelope of the maximal values)

(2) a waveform based on minimal values of the characteristic waveform (for example, a diagram obtained by connecting minimal values adjacent to each other or an envelope of the minimal values)

(3) a waveform based on middle points of distances in an amplitude direction between the waveform of (1) and the waveform of (2) (for example, a diagram obtained by connecting middle points adjacent to each other or a waveform formed by the middle points).

FIG. 4B illustrates an example of the waveforms of the above-described (1) to (3) that can be generated as the waveform for removing the base line variation in the characteristic waveform in S1035 in a case where the envelope of the minimal values (bottom) of the pulse wave is used as the characteristic waveform.

In S1037, the control unit 110 removes the base line variation in the characteristic waveform by subtracting the waveform generated in S1035 from the characteristic waveform. The control unit 110 stores the data of the characteristic waveform from which the base line variation is removed as the data of the respiration effort waveform in the memory 107. FIG. 4C illustrates the respiration effort waveform obtained by removing the base line variation from the characteristic waveform in a case where the characteristic waveform is the envelope of the minimal values (bottom) of the pulse wave and the above-described (3) (waveform formed by the middle points of the distances in the amplitude direction between the envelope of the maximal values and the envelope of the minimal values in the characteristic waveform) is generated as the waveform for removing the base line variation in the characteristic waveform in S1035.

Incidentally, the control unit 110 may remove the base line variation in the characteristic waveform by applying high-pass filter processing to the characteristic waveform in place of the processing of S1035 and S1037. However, because the pulse wave measured during sleep often includes abrupt base line variation, it is not easy to design a high-pass filter that removes the base line variation. Therefore, the method that generates the waveform representing the base line variation from the characteristic waveform of the pulse wave and that subtracts the waveform representing the base line variation from the characteristic waveform can remove the base line variation by simpler processing and with higher accuracy. Incidentally, of the waveforms (1) to (3) for removing the base line variation, (3) is considered to best represent the base line variation. It is therefore preferable to use the waveform (3) in a case where higher accuracy is desired from an increase in an amount of operation.

The control unit 110 continuously performs the above-described processing with pulse wave data corresponding to a predetermined continuous period of time as a processing unit. Incidentally, the control unit 110 may determine the interval of a next processing unit such that the interval partly overlaps an immediately preceding processing unit as in the calculation of a moving average. In this case, for an interval in which two respiration effort waveforms generated for the respective processing units overlap each other, one of the respiration effort waveforms may be used, or the respiration effort waveforms may be averaged.

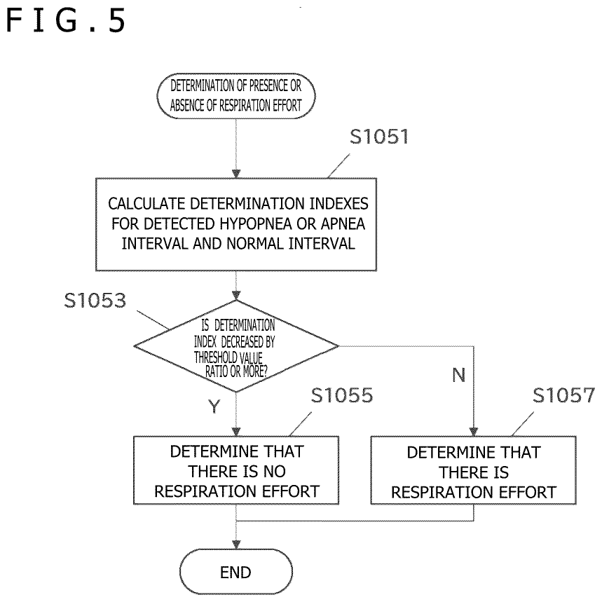

Next, details of the processing of determining the presence or absence of a respiration effort in S105 will be described with reference to a flowchart illustrated in FIG. 5.

In S1051, the control unit 110 calculates determination indexes with regard to the presence or absence of a respiration effort for a respiration effort waveform of a hypopnea or apnea interval for which a determination is to be made and a respiration effort waveform of a normal interval corresponding to an immediately preceding and/or immediately succeeding predetermined period of time, for example.

A determination index may be, for example, a maximum amplitude of a respiration effort waveform within an interval, a maximum area of a region enclosed by a base line of the respiration effort waveform and the respiration effort waveform, or the like. The base line of the respiration effort waveform can be defined by any publicly known method such as the application of a low-pass filter to the respiration effort waveform or an estimation from a middle point group of the amplitude of the respiration effort waveform. The control unit 110 stores the determination indexes calculated for the respective respiration effort waveforms in the memory 107.

In S1053, the control unit 110 determines whether or not the determination index for the hypopnea or apnea interval is decreased by a predetermined threshold value ratio or more from the determination index for the normal interval. The control unit 110 performs S1055 when determining that there is a decrease by the predetermined threshold value ratio or more. The control unit 110 performs S1057 when not determining that there is a decrease by the predetermined threshold value ratio or more.

Incidentally, in S1051, for the hypopnea or apnea interval, each amplitude of the respiration effort waveform within the interval or the area of each region enclosed by the base line of the respiration effort waveform and the respiration effort waveform may be calculated as the determination index. In this case, in S1053, the control unit 110 detects determination indexes decreased by the predetermined threshold value ratio or more from the determination index for the normal interval from among a plurality of determination indexes calculated for the hypopnea or apnea interval, and determines whether or not a detection ratio of the determination indexes is equal to or higher than a threshold value. Then, the control unit 110 performs S1055 when determining that the ratio of the determination indexes decreased by the predetermined threshold value ratio or more is equal to or higher than the threshold value. The control unit 110 performs S1057 when not determining that the ratio of the determination indexes decreased by the predetermined threshold value ratio or more is equal to or higher than the threshold value.

In S1055, the control unit 110 determines that there is no respiration effort in the detected hypopnea or apnea interval. The control unit 110 then ends the processing of determining the presence or absence of a respiration effort.

In S1057, the control unit 110 determines that there is a respiration effort in the detected hypopnea or apnea interval. The control unit 110 then ends the processing of determining the presence or absence of a respiration effort.

Incidentally, a case of determining that there is a respiration effort or there is no respiration effort for the whole of the hypopnea or apnea interval has been described here. However, the control unit 110 may determine both the presence and absence of a respiration effort for one hypopnea or apnea interval.

For example, in S1051, for the hypopnea or apnea interval, the control unit 110 calculates, as the determination index, each amplitude of the respiration effort waveform within the interval (for example, the amplitude in each pulse or cycle in the respiration effort waveform within the interval) or the area of each region enclosed by the base line of the respiration effort waveform and the respiration effort waveform (for example, the area of a region enclosed by the base line and the respiration effort waveform in each pulse or cycle in the respiration effort waveform). Then, in S1053, the control unit 110 determines that an interval in which a predetermined number or more of determination indexes decreased by the predetermined threshold value ratio or more from the determination index for the normal interval continue among a plurality of determination indexes calculated for the hypopnea or apnea interval (for example, determination indexes calculated for each pulse or cycle in the hypopnea or apnea interval) is an interval without a respiration effort, and the control unit 110 determines that an interval in which a predetermined number or more of determination indexes not decreased by the predetermined threshold value ratio or more continues is an interval with a respiration effort. It is thereby possible to determine MSA.

Thus, according to the present embodiment, the type of respiration disorder in a hypopnea or apnea interval is identified on the basis of at least one of the pulse wave measured in a simple PSG test and SpO₂ or the respiration waveform. It is therefore possible to reduce a burden on the subject, and remedy the inability to determine the presence or absence of a respiration effort and a decrease in accuracy of the determination, which are caused by a displacement of the respiratory movement sensor due to inappropriate wearing of the respiratory movement sensor and body movement.

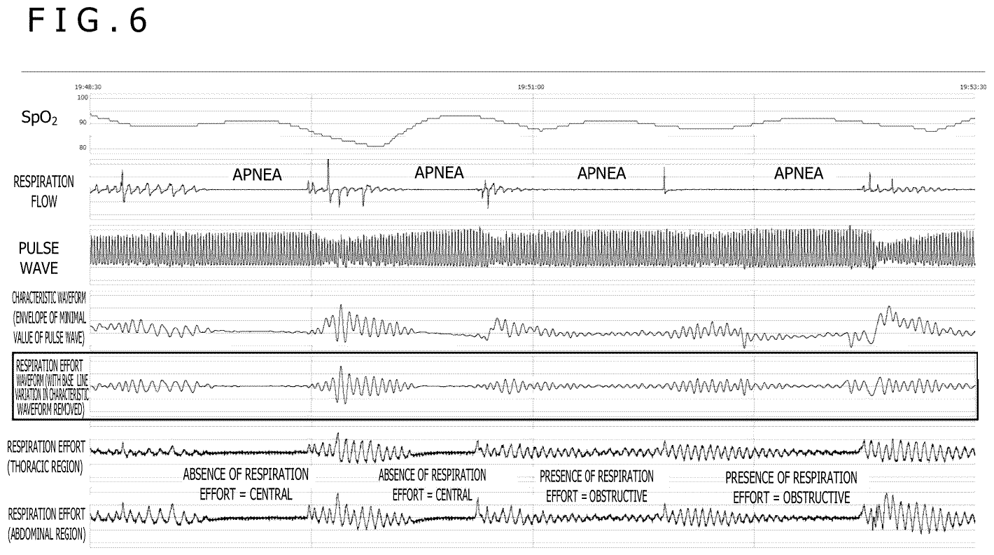

FIG. 6 is a diagram illustrating a specific example including a determination result of the present embodiment and signals obtained by the respiratory movement sensor properly worn on a thoracic region and an abdominal region. Four apnea intervals are detected on the basis of the respiration waveform (respiration flow). Further, on the basis of the respiration effort waveform generated by the method according to the present embodiment, it is determined that there is no respiration effort for the first two intervals, and it is determined that there is a respiration effort for the other two intervals. As a result, CSA is determined for the first two intervals, and OSA is determined for the other two intervals. In addition, it is understood that the respiration effort waveform generated by the method according to the present embodiment has a high similarity to the signals obtained by the respiratory movement sensor properly worn on the thoracic region and the abdominal region.

Incidentally, the biological information processing device according to the present disclosure can also be implemented as a program (application software) that makes a generally available general-purpose information processing device such as a smart phone or a tablet terminal perform the operations of the flowcharts illustrated in FIG. 2, FIG. 3, and FIG. 5. Hence, such a program and a storage medium storing the program (an optical recording medium such as a CD-ROM or a DVD-ROM, a magnetic recording medium such as a magnetic disk, a semiconductor memory card, and the like) also constitute the present disclosure.

The disclosure of the present embodiment includes a biological information processing device, a simple PSG test device, a biological information processing method, and a program in the following.

### (Item 1)

A biological information processing device including:
an obtainer configured to obtain a pulse wave during sleep;
a detector configured to detect a hypopnea or apnea interval in a period of measurement of the pulse wave;
a determiner configured to determine presence or absence of a respiration effort on the basis of the pulse wave in the hypopnea or apnea interval; and
an identifier configured to identify a type of respiration disorder for the hypopnea or apnea interval on the basis of the presence or absence of the respiration effort.

According to this item, it is possible to identify a type of sleep apnea for the hypopnea or apnea interval without using a respiratory movement sensor.

### (Item 2)

The biological information processing device according to item 1, in which
the determiner determines the presence or absence of the respiration effort on the basis of a characteristic waveform obtained on the basis of a characteristic point of the pulse wave.

According to this item, it is possible to determine the presence or absence of the respiration effort without using a respiratory movement sensor.

### (Item 3)

The biological information processing device according to item 2, in which
the characteristic waveform is a waveform based on any one of a characteristic point of an amplitude of the pulse wave, an amplitude value of the pulse wave, and a time interval of the pulse wave.

According to this item, it is possible to determine the presence or absence of the respiration effort without using a respiratory movement sensor.

### (Item 4)

The biological information processing device according to item 2 or 3, in which
the determiner determines the presence or absence of the respiration effort on the basis of a waveform obtained by removing base line variation in the characteristic waveform.

According to this item, it is possible to determine the presence or absence of the respiration effort with high accuracy.

### (Item 5)

The biological information processing device according to item 4, in which
the determiner removes the base line variation in the characteristic waveform by using a waveform obtained on the basis of the characteristic waveform or by filter processing.

According to this item, it is possible to remove the base line variation from the pulse wave in which abrupt base line variation can occur by simple processing and with high accuracy, and thus favorably determine the presence or absence of the respiration effort.

### (Item 6)

The biological information processing device according to item 5, in which
the waveform obtained on the basis of the characteristic waveform includes any one of a waveform based on a maximal value of the characteristic waveform, a waveform based on a minimal value of the characteristic waveform, a waveform based on a middle point of a distance in an amplitude direction between the waveform based on the maximal value of the characteristic waveform and the waveform based on the minimal value of the characteristic waveform, or a waveform obtained by applying filter processing to the characteristic waveform.

According to this item, it is possible to remove the base line variation from the pulse wave in which abrupt base line variation can occur by simple processing and with high accuracy, and thus favorably determine the presence or absence of the respiration effort.

### (Item 7)

The biological information processing device according to any one of items 4 to 6, in which
the determiner determines the presence or absence of the respiration effort in the hypopnea or apnea interval on the basis of an amplitude of the characteristic waveform from which the base line variation is removed or an area of a region enclosed by a base line and the characteristic waveform from which the base line variation is removed, for the hypopnea or apnea interval and a non-hypopnea or non-apnea interval.

According to this item, it is possible to determine the presence or absence of the respiration effort without using a respiratory movement sensor.

### (Item 8)

The biological information processing device according to item 7, in which
the determiner determines that there is no respiration effort in the hypopnea or apnea interval in a case in which a maximum or average amplitude of the characteristic waveform from which the base line variation is removed or a maximum or an average area of the region enclosed by the base line and the characteristic waveform from which the base line variation is removed is decreased in the hypopnea or apnea interval to a threshold value ratio or less from the non-hypopnea or non-apnea interval.

According to this item, it is possible to determine the presence or absence of the respiration effort without using a respiratory movement sensor.

### (Item 9)

The biological information processing device according to any one of items 1 to 8, in which
the obtainer further obtains at least either a respiration waveform or an arterial oxygen saturation in the period of measurement of the pulse wave, and
the detector detects the hypopnea or apnea interval on the basis of at least either a decrease in an amplitude of the respiration waveform or a decrease in the arterial oxygen saturation.

According to this item, it is possible to determine the hypopnea or apnea interval on the basis of the respiration waveform.

### (Item 10)

The biological information processing device according to any one of items 1 to 9, in which
the identifier
   identifies the type of the respiration disorder as obstructive sleep apnea for the hypopnea or apnea interval in which the determiner determines that there is a respiration effort, and
   identifies the type of the respiration disorder as central sleep apnea for the hypopnea or apnea interval in which the determiner determines that there is no respiration effort.

According to this item, it is possible to identify a type of sleep disorder for the hypopnea or apnea interval.

### (Item 11)

The biological information processing device according to item 10, in which
the determiner determines an interval with a respiration effort and an interval without a respiration effort in the hypopnea or apnea interval, and
the identifier identifies the type of the respiration disorder as mixed sleep apnea for the hypopnea or apnea interval including the interval in which the determiner determines that there is a respiration effort and the interval in which the determiner determines that there is no respiration effort.

According to this item, it is possible to identify a type of sleep disorder for the hypopnea or apnea interval.

### (Item 12)

A simple PSG test device including:
a section configured to obtain a pulse wave and an arterial oxygen saturation;
a section configured to obtain a respiration waveform; and
the biological information processing device according to any one of items 1 to 11.

According to this item, it is possible to provide a simple PSG test device that can identify a type of sleep apnea at a time of measurement without using a respiratory movement sensor.

### (Item 13)

A biological information processing method performed by a biological information processing device, the biological information processing method including:
obtaining a pulse wave during sleep;
detecting a hypopnea or apnea interval in a period of measurement of the pulse wave;
determining presence or absence of a respiration effort on the basis of the pulse wave in the hypopnea or apnea interval; and
identifying a type of respiration disorder for the hypopnea or apnea interval on the basis of the presence or absence of the respiration effort.

According to this item, it is possible to identify a type of sleep apnea for the hypopnea or apnea interval without using a respiratory movement sensor.

### (Item 14)

A non-transitory storage medium storing a program for making a computer function as each section possessed by the biological information processing device according to any one of items 1 to 11.

According to this item, a non-transitory storage medium storing a program for implementing the medical device that has the above-described effects is provided.

The present disclosure is not limited to the contents of the foregoing embodiment and is susceptible of various changes and modifications without departing from the spirit and scope of the disclosure. Hence, claims are attached to make public the scope of the disclosure.

What is claimed is:

1. A biological information processing device comprising:
an obtainer configured to obtain a pulse wave during sleep;
a detector configured to detect a hypopnea or apnea interval in a period of measurement of the pulse wave;
a determiner configured to determine presence or absence of a respiration effort on a basis of the pulse wave in the hypopnea or apnea interval; and
an identifier configured to identify a type of respiration disorder for the hypopnea or apnea interval on a basis of the presence or absence of the respiration effort.

2. The biological information processing device according to claim 1, wherein
the determiner determines the presence or absence of the respiration effort on a basis of a characteristic waveform obtained on a basis of a characteristic point of the pulse wave.

3. The biological information processing device according to claim 2, wherein
the characteristic waveform is a waveform based on any one of a characteristic point of an amplitude of the pulse wave, an amplitude value of the pulse wave, and a time interval of the pulse wave.

4. The biological information processing device according to claim 2, wherein
the determiner determines the presence or absence of the respiration effort on a basis of a waveform obtained by removing base line variation in the characteristic waveform.

5. The biological information processing device according to claim 4, wherein
the determiner removes the base line variation in the characteristic waveform by using a waveform obtained on a basis of the characteristic waveform or by filter processing.

6. The biological information processing device according to claim 5, wherein
the waveform obtained on the basis of the characteristic waveform includes any one of a waveform based on a maximal value of the characteristic waveform, a waveform based on a minimal value of the characteristic waveform, a waveform based on a middle point of a distance in an amplitude direction between the waveform based on the maximal value of the characteristic waveform and the waveform based on the minimal value of the characteristic waveform, or a waveform obtained by applying filter processing to the characteristic waveform.

7. The biological information processing device according to claim 4, wherein
the determiner determines the presence or absence of the respiration effort in the hypopnea or apnea interval on a basis of an amplitude of the characteristic waveform from which the base line variation is removed or an area of a region enclosed by a base line and the characteristic waveform from which the base line variation is removed, for the hypopnea or apnea interval and a non-hypopnea or non-apnea interval.

8. The biological information processing device according to claim 7, wherein
the determiner determines that there is no respiration effort in the hypopnea or apnea interval in a case in which a maximum or average amplitude of the characteristic waveform from which the base line variation is removed or a maximum or an average area of the region enclosed by the base line and the characteristic waveform from which the base line variation is removed is decreased in the hypopnea or apnea interval to a threshold value ratio or less from the non-hypopnea or non-apnea interval.

9. The biological information processing device according to claim 1, wherein
the obtainer further obtains at least either a respiration waveform or an arterial oxygen saturation in the period of measurement of the pulse wave, and
the detector detects the hypopnea or apnea interval on a basis of at least either a decrease in an amplitude of the respiration waveform or a decrease in the arterial oxygen saturation.

10. The biological information processing device according to claim 1, wherein
the identifier
identifies the type of the respiration disorder as obstructive sleep apnea for the hypopnea or apnea interval in which the determiner determines that there is a respiration effort, and
identifies the type of the respiration disorder as central sleep apnea for the hypopnea or apnea interval in which the determiner determines that there is no respiration effort.

11. The biological information processing device according to claim 10, wherein
the determiner determines an interval with a respiration effort and an interval without a respiration effort in the hypopnea or apnea interval, and
the identifier identifies the type of the respiration disorder as mixed sleep apnea for the hypopnea or apnea interval including the interval in which the determiner determines that there is a respiration effort and the interval in which the determiner determines that there is no respiration effort.

12. A simple polysomnography test device comprising:
a section configured to obtain a pulse wave and an arterial oxygen saturation;
a section configured to obtain a respiration waveform; and
the biological information processing device according to claim 1.

13. A biological information processing method performed by a biological information processing device, the biological information processing method comprising:
obtaining a pulse wave during sleep;
detecting a hypopnea or apnea interval in a period of measurement of the pulse wave;
determining presence or absence of a respiration effort on a basis of the pulse wave in the hypopnea or apnea interval; and
identifying a type of respiration disorder for the hypopnea or apnea interval on a basis of the presence or absence of the respiration effort.

14. A non-transitory storage medium storing a program for making a computer function as each section possessed by the biological information processing device according to claim 1.
